(19) United States
(12) Patent Application Publication
(10) Pub. No.: US 2008/0051872 A1
(43) Pub. Date: Feb. 28, 2008

(54) BIOCORRODIBLE METALLIC IMPLANT HAVING A COATING OR CAVITY FILLING MADE OF A PEG/PLGA COPOLYMER

(75) Inventor: Alexander Borck, Aurachtal (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/843,048

(22) Filed: Aug. 22, 2007

(30) Foreign Application Priority Data
Aug. 22, 2006 (DE) 10 2006 039 346.5

(51) Int. Cl. A61F 2/06; C08L 53/00

(52) U.S. Cl. 623/1.15; 523/121; 525/91

(57) ABSTRACT

A stent made of a biocorrodible metallic material having a coating or cavity filling comprising a diblock or triblock copolymer made of (i) a poly(D,L-lactide-co-glycolide) block and (ii) a polyethylene glycol block.

### PRIORITY CLAIM

This patent application claims priority to German Patent Application No. 10 2006 039 346.5, filed Aug. 22, 2006, the disclosure of which is incorporated herein by reference in its entirety.

### FIELD

The present disclosure relates to an implant made of a biocorrodible metallic material, which has a coating or cavity filling comprising a polyethylene glycol/poly(D,L-lactide-co-glycolide) copolymer (PEG/PLGA copolymer), as well as a method for using the PEG/PLGA copolymer.

### BACKGROUND

Implants are used in modern medical technology in manifold embodiments. Implants are used, for example, for supporting vessels, hollow organs, and duct systems (endovascular implants), for attaching and temporarily fixing tissue implants and tissue transplants, and for orthopedic purposes, for example, as nails, plates, or screws.

Thus, for example, the implantation of stents has been established as one of the most effective therapeutic measures in the treatment of vascular illnesses. Stents provide a support function in the hollow organs of a patient. Stents of typical construction have a filigree support structure made of metallic struts for this purpose, which is first provided in a compressed form for introduction into the body and is expanded at the location of application. One of the main areas of application of such stents is permanently or temporarily expanding and keeping open vascular constrictions, in particular, constrictions (stenoses) of the coronary vessels. In addition, for example, aneurysm stents are also known, which are used to support damaged vascular walls.

Stents have a peripheral wall of sufficient supporting force to keep the constricted vessel open to the desired degree and a tubular main body through which the blood flow continues to run unimpeded. The supporting peripheral wall is frequently implemented as a latticed structure, which allows the stent to be inserted in a compressed state having a small external diameter up to the constriction point of the particular vessel to be treated and to be expanded there with the aid of a balloon catheter, for example, enough that the vessel has the desired, enlarged internal diameter. To avoid unnecessary vascular damage, the stent should not elastically recoil at all or, in any case, should elastically recoil only slightly after the expansion and removal of the balloon, so that the stent only has to be expanded slightly beyond the desired final diameter upon expansion. Further criteria which are desirable in a stent include, but are not limited to, for example, uniform area coverage and a structure which allows a specific flexibility in relation to the longitudinal axis of the stent. In practice, the stent is typically molded from a metallic material to implement the cited mechanical properties.

In addition to the mechanical properties of a stent, the stent should comprise a biocompatible material to avoid rejection reactions. Currently, stents are used in approximately 70% of all percutaneous interventions; however, an in-stent restenosis occurs in 25% of all cases because of excess neointimal growth, which is caused by a strong proliferation of the arterial smooth muscle cells and a chronic inflammation reaction. Various solution approaches are followed to reduce the restenosis rate.

One approach for reducing the restenosis rate includes providing a pharmaceutically active substance (active ingredient) on the stent, which counteracts the mechanisms of restenosis and supports the course of healing. The active ingredient is applied in pure form or embedded in a carrier matrix as a coating or filled in cavities of the implant. Examples comprise the active ingredients sirolimus and paclitaxel.

A further, more promising approach for solving the problem is the use of biocorrodible materials and their alloys because, typically, a permanent support function by the stent is not necessary; the initially damaged body tissue regenerates. Thus, it is suggested in German Patent Application No. 197 31 021 A1 that medical implants be molded from a metallic material whose main component is iron, zinc, or aluminum or an element from the group consisting of alkali metals or alkaline earth metals. Alloys based on magnesium, iron, and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc, iron and the like. Furthermore, the use of a biocorrodible magnesium alloy having a proportion of magnesium >90%, yttrium 3.7-5.5%, rare earth metals 1.5-4.4%, and the remainder <1% is known from German Patent Application No. 102 53 634 A1, which is suitable, in particular, for producing an endoprosthesis, e.g., in the form of a self-expanding or balloon-expandable stent. The use of biocorrodible metallic materials in implants may result in a significant reduction of rejection or inflammation reactions.

The combination of active ingredient release and biocorrodible metallic material appears particularly promising. The active ingredient is applied as a coating or introduced into a cavity in an implant, usually embedded in a carrier matrix. For example, stents made of a biocorrodible magnesium alloy having a coating made of a poly(L-lactide) are known in the art. However, the following problems remain, in spite of the progress achieved.

The degradation products of the carrier matrix should not have any noticeable influence on the local pH value to avoid undesired tissue reactions, on one hand, and to reduce the influence on the corrosion process of the metallic implant material, on the other hand.

### SUMMARY

The present disclosure describes several exemplary embodiments of the present invention.

One aspect of the present disclosure provides an implant made of a biocorrodible metallic material, the implant comprising a coating or cavity filling comprising a diblock or triblock copolymer made of (i) a poly(D,L-lactide-co-glycolide) block and (ii) a polyethylene glycol block.

Another aspect of the present disclosure provides a method for coating a stent made of a biocorrodible metallic material, comprising a) producing a coating comprising a diblock or triblock copolymer made of a poly(D,L-lactifde-co-glycolide) block and a polyethylene glycol block and b) coating the stent with the coating. A further aspect of the present disclosure provides a method for filling a cavity in a stent made of a biocorrodible metallic material, comprising a) producing a filling comprising a diblock or triblock copolymer made of a poly(D,L-lactifde-co-glycolide) block and a polyethylene glycol block and b) filling the cavity with the filling.

### DETAILED DESCRIPTION

A first aspect of the present disclosure provides an implant made of a biocorrodible metallic material having a coating or cavity filling comprising a diblock or triblock copolymer made of (i) a poly(D,L-lactide-co-glycolide) block, and (ii) a polyethylene glycol block.

The PEG/PLGA copolymer displays initial degradation in the polyethylene glycol block. The poly(D,L-lactide-co-glycolide) block is significantly more stable to degradation. During the degradation, hydroxy groups arise, which have a slight effect on the local pH value because of their chemical nature, however. In contrast, a carrier matrix made of polylactide hydrolyzes while forming acid functions, which are responsible for tissue reactions, such as inflammation. In addition to the positive influence on the tissue, hydroxyl groups are more suitable for the main body, in particular, if the hydroxyl group comprises magnesium and its alloys, because magnesium and its alloys do not additionally accelerate the degradation.

The rapid degradation of the polyethylene glycol block also results in a significant increase of the porosity of the carrier matrix, so that the degradation of the biocorrodible metallic implant material is influenced less by the presence of the carrier matrix.

For purposes of the present disclosure, a coating is an at least partial application of the components to the main body of the implant, in particular, a stent. Preferably, the entire surface of the main body of the implant or stent is covered by the coating. Alternatively, the PEG/PLGA copolymer may be provided in a cavity of the implant or stent.

The PEG/PLGA copolymer used in the present disclosure is highly biocompatible and biodegradable. The processing of the PEG/PLGA copolymer may be performed according to standard methods. The block copolymer has a hydrophobic domain and a hydrophilic domain and is capable of absorbing hydrophobic and hydrophilic materials. Materials having amphiphilic characteristics may also be solubilized in this matrix. The carrier matrix is, therefore, preferably suitable for incorporating active ingredients which change their solution properties upon a change of the pH value (e.g., active ingredients having amine functions); a problem which occurs, in particular, upon the degradation of magnesium alloys. The copolymer is also pH-value-neutral, so that the material is especially suitable for embedding pH sensitive active ingredients. The PEG/PLGA copolymer is, therefore, typically used as a carrier matrix for a pharmaceutical active ingredient, but may also contain fluorescence or x-ray markers or other additives, if necessary. Diblock and triblock copolymers of PEG/PLGA are commercially available under the trade name RESOMER™ from Boehringer Ingelheim, Germany.

The polyethylene glycol block preferably has a mean molecular weight in the range from 4,000 to 8,000 Dalton.

Furthermore, it is preferable if the poly(D,L-lactide-co-glycolide) block has a mean molecular weight in the range from 20,000 to 120,000 Dalton.

For purposes of the present disclosure, the term “biocorrodible” is used for metallic materials in which degradation occurs in physiological surroundings which finally results in the entire implant or the part of the implant made of the material losing its mechanical integrity. For purposes of the present disclosure, biocorrodible metallic materials particularly comprise metals and alloys selected from the group of elements consisting of iron, tungsten, and magnesium. For purposes of the present disclosure, an alloy is a metallic microstructure whose main component is magnesium, iron, or tungsten. The main component is the alloy component whose weight proportion in the alloy is highest. A proportion of the main component is preferably more than 50 wt.-% (weight-percent), in particular, more than 70 wt.-%.

The biocorrodible material is preferably a magnesium alloy. In particular, the biocorrodible magnesium alloy contains yttrium and further rare earth metals, because an alloy of this type is distinguished on the basis of its physiochemical properties and high biocompatibility, in particular, also its degradation products.

A magnesium alloy of the composition rare earth metals 5.2-9.9 wt.-%, yttrium 3.7-5.5 wt.-%, and the remainder <1 wt.-% is especially preferable, magnesium making up the proportion of the alloy to 100 wt.-%. This magnesium alloy has already confirmed its special suitability experimentally and in initial clinical trials, i.e., it displays a high biocompatibility, favorable processing properties, good mechanical characteristics, and corrosion behavior adequate for the intended uses. For purposes of the present disclosure, the collective term “rare earth metals” includes scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The metallic materials and/or magnesium alloys are to be selected in their composition in such a way that they are biocorrodible. Artificial plasma, as has been previously described according to EN ISO 10993-15:2000 for biocorrosion assays (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as a testing medium for testing the corrosion behavior of an alloy coming into consideration. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces in a known way. The artificial plasma according to EN ISO 10993-15:2000 corresponds to a medium similar to blood and thus represents a possibility for simulating a reproducible physiological environment.

For purposes of the present disclosure, implants are devices introduced into the body via a surgical method and comprise fasteners for bones, such as screws, plates, or nails, surgical suture material, intestinal clamps, vascular clips, prostheses in the area of the hard and soft tissue, and anchoring elements for electrodes, in particular, of pacemakers or defibrillators.

The implant is preferably a stent. Stents of typical construction have filigree support structures made of metallic struts which are initially provided in an unexpanded state for introduction into the body and are then widened into an expanded state at the location of application.

A second aspect of the present disclosure relates to a method for using PEG/PLGA copolymers of the composition described above as a coating material for a stent made of a biocorrodible metallic material or as a filling for a cavity in a stent made of a biocorrodible metallic material.

### EXAMPLE

Stents made of the biocorrodible magnesium alloy WE43 (97 wt.-% magnesium, 4 wt.-% yttrium, 3 wt.-% rare earth metals besides yttrium) were coated as follows:

The magnesium surfaces of the stent were roughened by treatment using an argon plasma to achieve greater adhesion of the active ingredient on the stent surface. Alternatively or additionally, a surface modification, e.g., by silanization using methoxy or epoxy silanes or with the aid of phosphonic acid derivatives, may increase the adhesion capability to the metallic main body.

A 0.1% solution of the block copolymer (diblock copolymer made of poly(D,L-lactide-co-glycolide) block and 15% polyethylene glycol block (5000 Dalton); available for purchase under the trade name RESOMER™, type RGP d 50155 from Boehringer Ingelheim, Germany) in chloroform was used. The solution was sprayed on the stent using an airbrush system and then dried for 24 hours at room temperature.

All patents, patent applications and publications referred to herein are incorporated by reference in their entirety.

What is claimed is:

1. An implant made of a biocorrodible metallic material, the implant comprising:
a) a coating or cavity filling comprising a diblock or triblock copolymer comprising (i) a poly(D,L-lactide-co-glycolide) block; and (ii) a polyethylene glycol block.

2. The implant of claim 1, wherein the biocorrodible metallic material is a magnesium alloy.

3. The implant of claim 1, wherein the polyethylene glycol block has a mean molecular weight in the range from 4,000 to 8,000 Dalton.

4. The implant of claim 1, wherein the poly(D,L-lactide-co-glycolide) block has a mean molecular weight in the range from 20,000 to 120,000 Dalton.

5. The implant of claim 1, wherein the implant is a stent.

6. A method for coating a stent made of a biocorrodible metallic material, comprising:
a) producing a coating comprising a diblock or triblock copolymer made of a poly(D,L-lactifde-co-glycolide) block and a polyethylene glycol block; and
b) coating the stent with the coating of step a).

7. A method for filling a cavity in a stent made of a biocorrodible metallic material, comprising:
a) producing a filling comprising a diblock or triblock copolymer made of a poly(D,L-lactifde-co-glycolide) block and a polyethylene glycol block; and
b) filling the cavity with the filling of step a).

8. The implant of claim 2, wherein the polyethylene glycol block has a mean molecular weight in the range from about 4,000 to 8,000 Dalton.

9. The implant of claim 2, wherein the poly(D,L-lactide-co-glycolide) block has a mean molecular weight in the range from about 20,000 to 120,000 Dalton.

10. The implant of claim 3, wherein the poly(D,L-lactide-co-glycolide) block has a mean molecular weight in the range from about 20,000 to 120,000 Dalton.
